(19) United States
(12) Patent Application Publication
(10) Pub. No.: US 2021/0358245 A1
(43) Pub. Date: Nov. 18, 2021

(54) MEDICAL CONDITION VERIFICATION WITH BIOMETRIC CAPABILITY AND METHODS OF USING SAME

(71) Applicant: Rosina Samadani, Westport, CT (US)

(72) Inventor: Rosina Samadani, Westport, CT (US)

(21) Appl. No.: 17/322,373

(22) Filed: May 17, 2021

Related U.S. Application Data
(60) Provisional application No. 63/025,917, filed on May 15, 2020.

(51) Int. Cl. G07C 9/26; G06Q 50/26; G16H 40/67; G16H 10/65; G16H 10/40; G07C 9/29; H04B 1/3827; G06K 9/00; G01N 33/569

(52) U.S. Cl. CPC G07C 9/26; G06Q 50/265; G16H 40/67; G16H 10/65; G01N 33/56983; G07C 9/29; H04B 1/385; G06K 9/00087; G06K 9/00288; G16H 10/40

(57) ABSTRACT

A method of gatekeeping at an event includes providing a wearable electronic device including a processor, a memory, at least one biometric sensor and an indicator, validating the identity of an individual through the at least one biometric sensor, and confirming at least one criteria for entry, the criteria including, for the example of a virus, at least one of (1) the presence of antibodies corresponding to a virus, (2) a record of vaccination, or (3) a recent negative test for the virus within a predetermined period of time, signaling the condition of the individual via the indicator, presenting the indicator at a checkpoint, and admitting or refusing entry to the individual based on the indicator.

### CROSS-REFERENCE OF RELATED APPLICATIONS

This application claims priority to U.S. Provisional Application Ser. No. 63/025,917, filed May 15, 2020, the content of which are hereby incorporated by reference in its entirety as if fully set forth herein.

### FIELD OF THE DISCLOSURE

The present disclosure relates to access and verification of medical conditions and/or records. More particularly, the present disclosure relates to user-carried devices that permit access and verification to allow entry to events or venues, or for travel.

### BACKGROUND OF THE DISCLOSURE

Immediate access and verification of medical information would be helpful in many settings. For example, infectious diseases and viruses present difficult problems for social activities that require congregations of participants. For example, sporting events such as soccer games may function as “super spreader” events where a large number of people are infected at one time due to the large number of attendees, and their close proximity. Other activities such as travel, work or school and events, such as at schools, places of worship, and the workplace, also present unique challenges. During pandemics, many of these activities or events are cancelled because is difficult to ascertain or identify the infected individuals who, while asymptomatic, may nevertheless spread the infection. Thus, it would be beneficial to provide methods and systems to identify and verify an individual's medical condition, medical history or well-being before permitting the individual access to a venue.

### SUMMARY OF THE DISCLOSURE

In some examples, method of gatekeeping at an event comprises providing a wearable electronic device including a processor, a memory, at least one biometric sensor and an indicator, validating the identity of an individual through the at least one biometric sensor, and confirming at least one criteria for entry, the criteria including, for the example of a virus, at least one of (1) the presence of antibodies corresponding to a virus, (2) a record of vaccination, or (3) a recent negative test for the virus within a predetermined period of time, signaling the condition of the individual via the indicator, presenting the indicator at a checkpoint, and admitting or refusing entry to the individual based on the indicator.

### BRIEF DESCRIPTION OF THE DISCLOSURE

Various embodiments of the presently disclosed devices, systems and methods are disclosed herein with reference to the drawings, wherein:

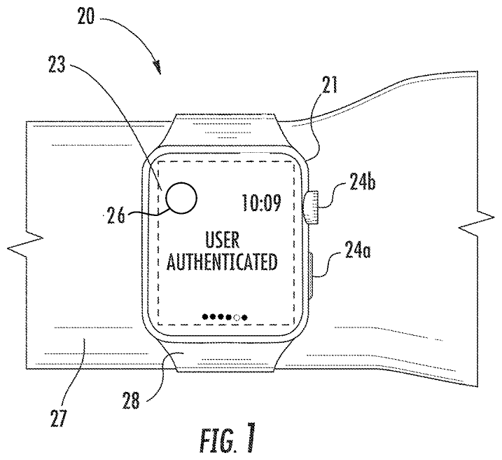

FIG. 1 is a schematic perspective view of a smartwatch having an indicator;

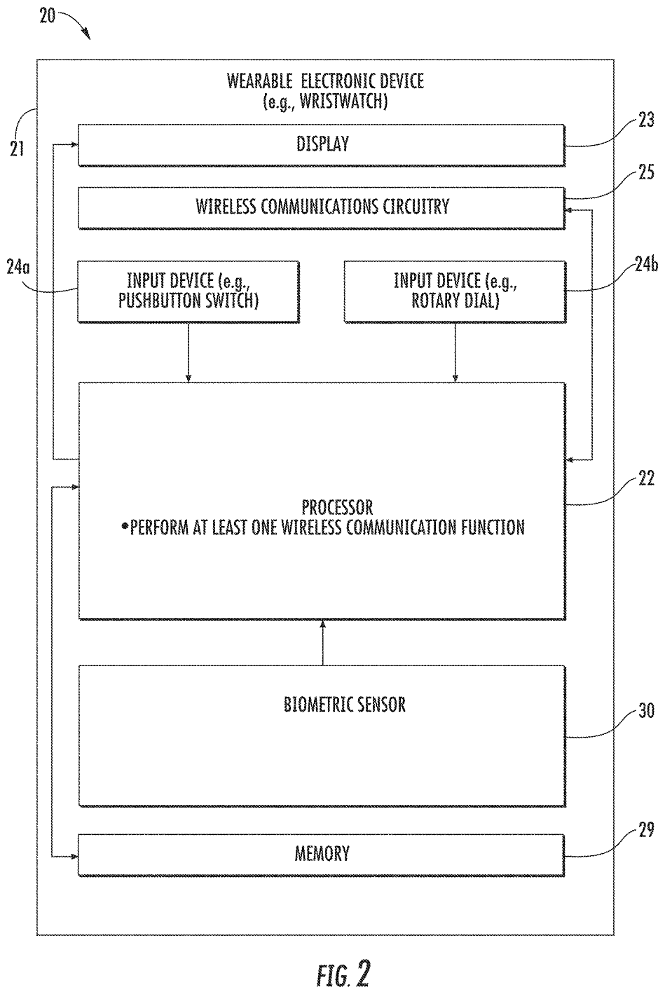

FIG. 2 is a schematic showing several modules of the smartwatch;

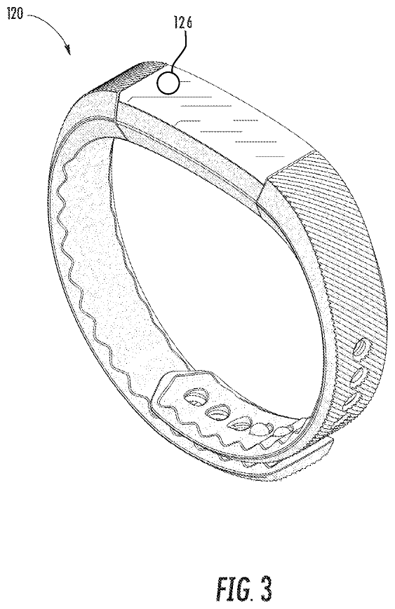

FIG. 3 is a schematic perspective view of a fitness tracker having an indicator;

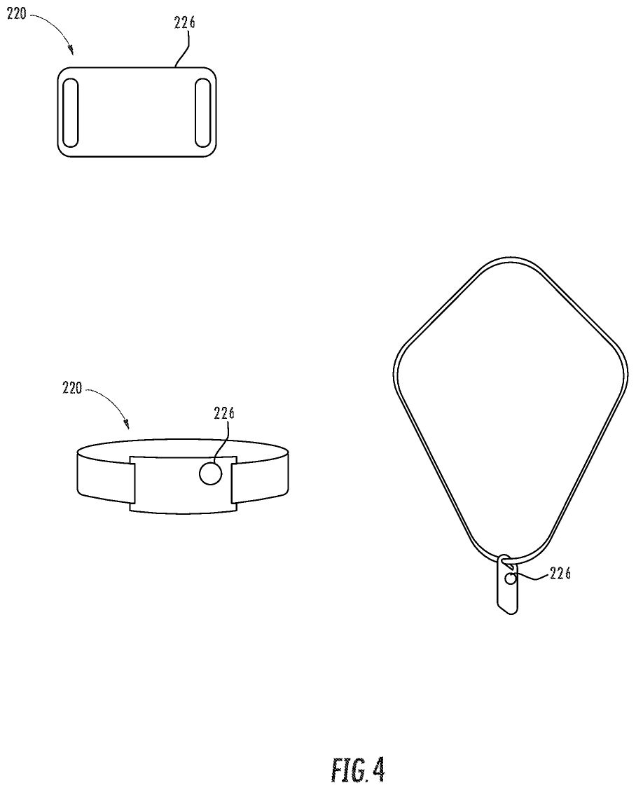

FIG. 4 is a schematic perspective view of a standalone tag coupleable to a necklace or bracelet;

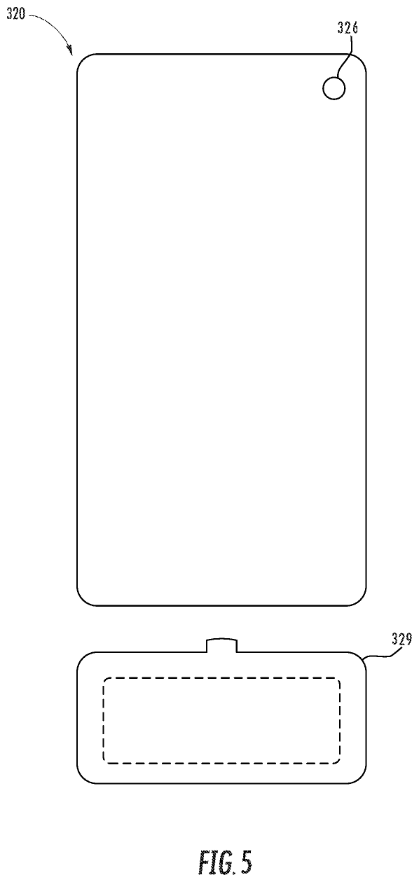

FIG. 5 is a schematic perspective view of a peripheral device for use with a smartphone.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to medical verification systems, conventional systems and devices suffer from some shortcomings as described above.

There therefore is a need for further improvements to the devices, systems, and methods of verifying medical conditions, medical history, and/or individual well-being. Among other advantages, the present disclosure may address one or more of these needs.

In some examples, the methods disclosed present a method for admitting attendees to an event (e.g., academic or exercise class, sporting event, concert, religious ceremony, party, conference, etc.) or venue (e.g., workplace, school, airport, train station) based on their medical history, well-being and/or condition. Specifically, the user may be prompted to verify a medical condition prior to being permitted to enter a setting. In the context of infectious diseases, such as viruses, a user may be prompted to verify that they are (1) vaccinated, (2) carriers of antibodies, for example, from a previous infection, and/or (3) are currently testing negative for a medical condition, such as the viral infection. Upon verifying that the user meets at least one of these conditions, the user may be permitted to enter a setting or facility.

Users are unlikely to carry their medical records or other confidential documents on a daily basis as these documents may be lost. Additionally, a system of verifying medical conditions through reading of documents would be slow and untenable, raising both effectiveness and privacy concerns. Moreover, a traditional system may be circumvented, allowing infected individuals to harm other attendees.

Conversely, biometric sensing and matching is a reliable and widely-used technique for personal identification or verification. For example, a common approach to biometric identification involves scanning a sample fingerprint or an image thereof and storing the image and/or unique characteristics of the fingerprint image. The characteristics of a sample fingerprint may be compared to information for reference fingerprints already in a database to determine proper identification a person, such as for verification purposes. Similar techniques may be used with iris or retina recognition, face recognition, voice recognition, or saliva microbiome, or a combination thereof.

A biometric sensor may be particularly advantageous for verification and/or authentication in an electronic device, and more particularly, a portable device. Such a biometric sensor may be carried by the body of a portable electronic device, for example, and may be sized to sense a biometric, for example, a fingerprint from a single-finger or any other biometrics discussed above. Where a biometric sensor is integrated into an electronic device or host device as noted above, it may be desirable to perform authentication, particularly performing another task or an application on the electronic device.

Referring initially to FIGS. 1-2, a wearable electronic device 20 illustratively includes a device body 21 and a processor 22 carried on the device body. The electronic device 20 is illustratively a mobile wireless communications device, for example, a wearable wireless communications device, and includes a device band 28 or strap for securing to a wrist 27 of a user. The wearable electronic device 20 may be another type of wearable electronic device, as will be appreciated by those skilled in the art and further discussed below.

Wireless communications circuitry 25 (e.g. cellular, WLAN Bluetooth, etc.) is also carried within the device body 21 and coupled to the processor 22. The wireless communications circuitry 25 cooperates with the processor 22 to perform at least one wireless communications function, for example, for voice and/or data. In some embodiments, the wearable electronic device 20 may not include wireless communications circuitry 25.

A display 23 is also carried by the device body 21 and is coupled to the processor 22. The display 23 may be a liquid crystal display (LCD), for example, or may be another type of display, as will be appreciated by those skilled in the art. The wearable electronic device 20 may function as a wrist-watch, for example, such that the processor 22 cooperates with the display 23 to display a current time and other data. The processor 22 may cooperate with the display 23 to display other types of information, for example, application or communications notifications, movement-based data, biometric data, etc. The device 20 may also include a memory 29.

Finger-operated user input devices 24a, 24b, illustratively in the form of a pushbutton switch and a rotary dial are also carried by the device body 21 and are coupled to the processor 22. The pushbutton switch 24a and the rotary dial 24b cooperate with the processor 22 to perform a device function in response to operation thereof. For example, a device function may include a powering on or off of the wearable electronic device 20, initiating communication via the wireless communications circuitry 25, and/or performing a menu function. In at least some examples, the electronic device 20 include an indicator 26 (e.g., a visual indicator) on the display for verifying a medical record. The visual indicator 26 may be in the form of a circle that illuminates green or red based on the presence or absence of a given medical condition.

One possible method of using the device for medical verification will now be described. First the user may initialize the electronic device 20 with any one or a combination of the disclosed biometric features (e.g., face recognition, fingerprint, saliva sample, etc.) to link the device to a particular user. The user may then perform regular testing for a medical condition over predetermined intervals of time. For example, the user may perform tests for a given virus each day, or every 12-hour period. This diagnostic test may check for the presence of the virus or a side effect (e.g., increased temperature, irregular heartbeat, oxygen saturation, etc.), or for antibodies, or for proof of a vaccination. In at least some examples, such tests are performed with a separate test, either user-administered or at a testing facility, and the information is stored in a remote database and communicated to the electronic device 20. In at least some other examples, the test is integrated into the electronic device 20. Biometric sensing may be used to ensure that the wearer is indeed the owner of the device, and also the same user who has been tested.

One example of using the system will now be described. The user may begin by initializing the device as described above to the link the device to a specific identity. In some examples, each device may be linked to one unique user, and subsequent verification of the user's identity may be made using the biometric information. In use, a owner of the device may approach a venue to enter (e.g., a sporting event, classroom, conference, public transport, or workplace), and may be stopped at a checkpoint. The user may be interrogated for the status of a viral condition, and the visual indicator 26 may illuminate green if it is determined that the user is (1) vaccinated for the given virus, (2) is a carrier of antibodies that correlate to the virus, and/or (3) has tested for the virus in a recent period (e.g., over the past 12 hours). Specifically, while at the entrance of the venue or within a short distance of the venue, the electronic device 20 may first use its biometric capabilities to identify the user, communicate wirelessly with a remote server or database(s) and obtain the identified user's records, and present the visual indicator that the user meets one or more of the criteria for the medical condition to be deemed safe. The visual indicator may be seen and the user granted permission to enter the setting. Conversely, if the user's records do not show that one or more of the criteria are met to be deemed safe, the visual indicator may illuminate red and the user may be turned away. It will be understood that the visual indicator may instead be in the form of illuminating the entire display green or red, depending on whether passage will be granted. Alternatively, the visual indicator may include text such as “ENTER” or “NOT VERIFIED” in addition to, or instead of, a color. Instead of, or in addition to, a visual indicator, an audible beep or chime may also be used to signal the status of the individual to permit entry. Subsequent biometric sensing may be performed to ensure that the same user continues to carry the electronic device. In at least some examples, a biometric sensing may be performed at each checkpoint, or each time that an indicator is prompted or required for entry.

While the methods and systems have been described in connection with a wrist watch, it will be understood that a similar system and method may be used with a fitness tracker 120 having an indicator 120 (FIG. 3), or a standalone tag 220 may include some or all of these features, the tag being coupleable to a bracelet or necklace and having an indicator 226 (FIG. 4). In at least some examples, the tag includes an indicator such as a light, a display, a sound, or other feature to confirm the status of an individual as described above. Alternatively, the electronic device may be a cellular phone 320 having an indicator 326 capable of functioning as described above. Optionally, the cellular phone 320 may have a coupleable peripheral testing device 329 that is capable of collecting a biological sample from the user, such as a saliva or blood sample. The testing device 329 may be coupleable to the cellular phone 320 through one or more ports or by wirelessly communicating with the device 320. In some examples, the testing may be performed locally on the device without having to communicate with an external database to retrieve medical records.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure, including the use of data encryption and security methods that meets or exceeds industry or government standards. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding an securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates embodiments in which users selectively block the use of, or access to, personal information data, including biometric data, and further contemplates user restrictions on storing data in cloud-based services and/or restricting access to the same. That is, the present disclosure contemplates that hardware and/or software elements can be provided to prevent or block access to such personal information data.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A method of gatekeeping at an event, comprising:
providing a wearable electronic device including a processor, a memory, at least one biometric sensor and an indicator;
validating the identity of an individual through the at least one biometric sensor;
confirming at least one criteria for entry, the criteria including at least one of (1) the presence of antibodies corresponding to a virus, (2) a record of vaccination, or (3) a recent negative test for the virus within a predetermined period of time;
signaling the condition of the individual via the indicator;
presenting the indicator at a checkpoint; and
admitting or refusing entry to the individual based on the indicator.

2. The method of claim 1, wherein confirming the at least one criteria comprises communicating with a remote database.

3. The method of claim 1, wherein the indicator is a visual indicator.

4. The method of claim 1, wherein the indicator is an auditory indicator.

5. The method of claim 1, wherein the at least one biometric sensor comprises facial recognition.

6. The method of claim 1, wherein the at least one biometric sensor comprises a fingerprint.

7. The method of claim 1, wherein the at least one biometric sensor comprises a saliva-based biometric.

8. The method of claim 1, wherein the predetermined period is 12 hours.

9. A method of gatekeeping at an event, comprising:
providing a wearable electronic device including a processor, a memory, at least one biometric sensor, a diagnostic test, and an indicator;
validating the identity of an individual through the at least one biometric sensor;
performing the diagnostic test at a checkpoint;
confirming at least one criteria for entry, the criteria including at least one of (1) the presence of antibodies corresponding to a virus from the diagnostic test, or (2) a negative test for the virus from the diagnostic test;
signaling the condition of the individual via the indicator;
presenting the indicator at a checkpoint; and
admitting or refusing entry to the individual based on the indicator.

10. The method of claim 9, wherein the at least one biometric sensor and the diagnostic test are processed from a same sample.
